(19) United States
(12) Patent Application Publication
(10) Pub. No.: US 2025/0295343 A1
(43) Pub. Date: Sep. 25, 2025

(54) RETRACTABLE ECG LEAD DEVICE

(71) Applicants: Joshua Fitzgerald, Ganado, TX (US); Justin Sickle, Richmond, TX (US)

(72) Inventors: Joshua Fitzgerald, Ganado, TX (US); Justin Sickle, Richmond, TX (US)

(21) Appl. No.: 18/609,886

(22) Filed: Mar. 19, 2024

(51) Int. Cl. A61B 5/271; A61B 5/28; A61B 5/318; B65H 75/42

(52) U.S. Cl. CPC A61B 5/271; A61B 5/28; A61B 5/318; B65H 75/42; A61B 2562/222

(57) ABSTRACT

A retractable electrocardiogram (ECG) lead device electrically connected to an ECG machine, the retractable ECG lead device including a main body, a spool rod disposed within the main body, a plurality of lead spools rotatably disposed on the spool rod within the main body, the plurality of lead spools each including a lead spool wire wrapped around a center portion of the lead spool, and a lead spool lead connected to a portion of the lead spool wire disposed outside of the main body.

### BACKGROUND

### 1. Field

The present general inventive concept relates generally to an ECG lead device, and particularly, to a retractable ECG lead device.

### 2. Description of the Related Art

Electrocardiograms (ECGs or EKGs) are commonly used by healthcare professionals, medical professionals, and first responders, in order to measure electrical activity of a patient's heart to detect cardiac problems.

These ECGs often have many leads and wires that oftentimes become tangled during use and storage.

Therefore, there is a need for a convenient device that allows for easy retrieval and equally-easy storage of ECG leads.

### SUMMARY

The present general inventive concept provides a system to alert a retractable ECG lead device.

Additional features and utilities of the present general inventive concept will be set forth in part in the description which follows and, in part, will be obvious from the description, or may be learned by practice of the general inventive concept.

The foregoing and/or other features and utilities of the present general inventive concept may be achieved by providing a retractable electrocardiogram (ECG) lead device, including a retractable electrocardiogram (ECG) lead device electrically connected to an ECG machine, the retractable ECG lead device including a main body, a spool rod disposed within the main body, a left arm lead spool rotatably disposed on the spool rod within the main body, the left arm lead spool including a left arm lead spool wire wrapped around a center portion of the left arm lead spool, and a left arm lead spool lead connected to a portion of the left arm lead spool wire disposed outside of the main body, a right arm lead spool rotatably disposed on the spool rod within the main body the right arm lead spool including a right arm lead spool wire wrapped around a center portion of the right arm lead spool, and a right arm lead spool lead connected to a portion of the right arm lead spool wire disposed outside of the main body, a left leg lead spool rotatably disposed on the spool rod within the main body, the left leg lead spool including a left leg lead spool wire wrapped around a center portion of the left leg lead spool, and a left leg lead spool lead connected to a portion of the left leg lead spool wire disposed outside of the main body, and a right leg lead spool rotatably disposed on the spool rod within the main body, the right leg lead spool including a right leg lead spool wire wrapped around a center portion of the right leg lead spool, and a right leg lead spool lead connected to a portion of the right leg lead spool wire disposed outside of the main body.

The retractable ECG lead device may further include a plurality of 12 channel ports disposed on the main body to provide the electrical connection between the retractable ECG lead device and the ECG machine when a plurality of 12 channel leads of the ECG machine are connected to the plurality of 12 channel ports.

The left arm lead spool wire may be pulled a first predetermined distance out from a first wire aperture of the main body, such that the left arm lead spool wire remains pulled out from the main body at the first predetermined distance, the right arm lead spool wire may be pulled a second predetermined distance out from a second wire aperture of the main body, such that the second arm lead spool wire remains pulled out from the main body at the second predetermined distance, the left leg lead spool wire may be pulled a third predetermined distance out from a third wire aperture of the main body, such that the left leg lead spool wire remains pulled out from the main body at the third predetermined distance, and the right leg lead spool wire may be pulled a fourth predetermined distance out from a fourth wire aperture of the main body, such that the right leg lead spool wire remains pulled out from the main body at the fourth predetermined distance.

The left arm lead spool wire may be retracted back into the main body and automatically wrapped around the center portion of the left arm lead spool in response to the left arm lead spool wire being pulled again when the left arm lead spool wire is already extended at the first predetermined distance, the right arm lead spool wire may be retracted back into the main body and automatically wrapped around the center portion of the right arm lead spool in response to the right arm lead spool wire being pulled again when the right arm lead spool wire is already extended at the second predetermined distance, the left leg lead spool wire may be retracted back into the main body and automatically wrapped around the center portion of the left leg lead spool in response to the left leg lead spool wire being pulled again when the left leg lead spool wire is already extended at the third predetermined distance, and the right leg lead spool wire may be retracted back into the main body and automatically wrapped around the center portion of the right leg lead spool in response to the right leg lead spool wire being pulled again when the right leg lead spool wire is already extended at the fourth predetermined distance.

### BRIEF DESCRIPTION OF THE DRAWINGS

These and/or other features and utilities of the present generally inventive concept will become apparent and more readily appreciated from the following description of the embodiments, taken in conjunction with the accompanying drawings of which:

FIG. 1 illustrates a retractable electrocardiogram (ECG) lead device, according to an exemplary embodiment of the present general inventive concept.

### DETAILED DESCRIPTION

Various example embodiments (a.k.a., exemplary embodiments) will now be described more fully with reference to the accompanying drawings in which some example embodiments are illustrated. In the figures, the thicknesses of lines, layers and/or regions may be exaggerated for clarity.

Accordingly, while example embodiments are capable of various modifications and alternative forms, embodiments thereof are shown by way of example in the figures and will herein be described in detail. It should be understood, however, that there is no intent to limit example embodiments to the particular forms disclosed, but on the contrary, example embodiments are to cover all modifications, equivalents, and alternatives falling within the scope of the disclosure. Like numbers refer to like/similar elements throughout the detailed description.

It is understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

The terminology used herein is for the purpose of describing particular embodiments only and is not intended to be limiting of example embodiments. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements and/or components, but do not preclude the presence or addition of one or more other features, integers, steps, operations, elements, components and/or groups thereof.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art. However, should the present disclosure give a specific meaning to a term deviating from a meaning commonly understood by one of ordinary skill, this meaning is to be taken into account in the specific context this definition is given herein.

### List of Components

Electrocardiogram (ECG) Machine 10

Electrocardiogram (ECG) 12-Channel Leads 11

Retractable Electrocardiogram (ECG) Lead Device 100

Main Body 110

Main Body Cover 111

Spool Rod Aperture 112

First Wire Aperture 113

Second Wire Aperture 114

Third Wire Aperture 115

Fourth Wire Aperture 116

Spool Rod 120

Spool Rod Release Button 121

Left Arm Lead Spool 130

Left Arm Lead Spool Wire 131

Left Arm Lead Spool Lead 132

Left Arm Lead Spool Wire Catch 133

Left Arm Lead Spool Retraction Spring 134

Right Arm Lead Spool 140

Right Arm Lead Spool Wire 141

Right Arm Lead Spool Lead 142

Right Arm Lead Spool Wire Catch 143

Right Arm Lead Spool Retraction Spring 144

Left Leg Lead Spool 150

Left Leg Lead Spool Wire 151

Left Leg Lead Spool Lead 152

Left Leg Lead Spool Wire Catch 153

Left Leg Lead Spool Retraction Spring 154

Right Leg Lead Spool 160

Right Leg Lead Spool Wire 161

Right Leg Lead Spool Lead 162

Right Leg Lead Spool Wire Catch 163

Right Leg Lead Spool Retraction Spring 164

12 Channel Port Housing 170

12 Channel Ports 171

12 Channel Port Housing Door 172

12 Channel Port Housing Door Springs 173

Electric Cable 180

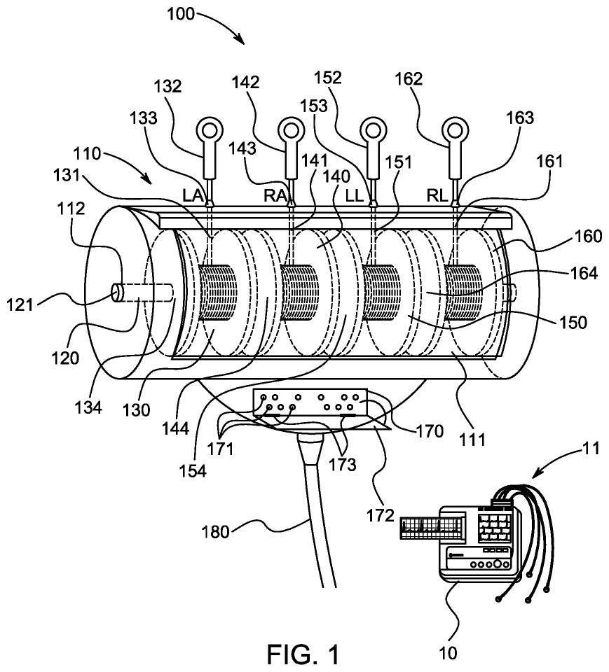

FIG. 1 illustrates a retractable electrocardiogram (ECG) lead device 100, according to an exemplary embodiment of the present general inventive concept.

The retractable ECG lead device 100, and all components therein and/or connected thereto, may be constructed from at least one of metal, plastic, wood, silicone, wiring, electronics, and rubber, etc., but is not limited thereto, and can be constructed from any material known to one of ordinary skill in the art.

Referring to FIG. 1, the retractable ECG lead device 100 may include a main body 110, a spool rod 120, a left arm lead spool 130, a right arm lead spool 140, a left leg lead spool 150, a right leg lead spool 160, a 12 channel port housing 170, and an electric cable 180, but is not limited thereto.

The main body 110 may include a main body cover 111, a spool rod aperture 112, a first wire aperture 113, a second wire aperture 114, a third wire aperture 115, and a fourth wire aperture 116, but is not limited thereto.

The spool rod 120 may include a spool rod release button 121, but is not limited thereto.

The spool rod 120 may be detachably disposed within the main body 110, such that the spool rod 120 is insertable through the spool rod aperture 112 and laterally within the main body 110. When the spool rod release button 121 is pressed, the spool rod 120 may be removed from the main body 110 through the spool rod aperture 112. When the spool rod 120 is removed from the main body 110, any of the left arm lead spool 130, the right arm lead spool 140, the left leg lead spool 150, and the right leg lead spool 160 may be removed from the main body 110.

When the main body cover 111 is open, thereby giving a user access to a inside portion of the main body 110, each of the left arm lead spool 130, the right arm lead spool 140, the left leg lead spool 150, and the right leg lead spool 160 may be inserted within the main body 110, and may be threaded onto the spool rod aperture 112 such that each of the left arm lead spool 130, the right arm lead spool 140, the left leg lead spool 150, and the right leg lead spool 160 may rotate along the spool rod 120.

When the spool rod release button 121 is pressed to allow the spool rod 120 to be removed from the main body 110 through the spool rod aperture 112, any of the left arm lead spool 130, the right arm lead spool 140, the left leg lead spool 150, and the right leg lead spool 160 may also be removed from the main body 110.

For ease of description, it will be assumed that each of the left arm lead spool 130, the right arm lead spool 140, the left leg lead spool 150, and the right leg lead spool 160 are disposed within the main body 110 to be rotatable about the spool rod 120. Alternatively, the spool rod 120 may itself be rotatable.

The left arm lead spool 130 may include a left arm lead spool wire 131, a left arm lead spool lead 132, a left arm lead spool wire catch 133, and a left arm lead spool retraction spring 134, but is not limited thereto.

The left arm lead spool wire 131 may be wrapped around a center portion of the left arm lead spool 130, such that a portion of the left arm lead spool wire 131 is threaded through the first wire aperture 112 that is disposed at a top portion of the main body 110. The left arm lead spool lead 132 may be disposed at an end of the left arm lead spool wire 131 that is disposed outside of the main body 110, and may be held in place by the left arm lead spool wire catch 133. A user may pull on the left arm lead spool lead 132 to cause the left arm lead spool 130 to rotate in a first direction about the spool rod 120, until the left arm lead spool wire 131 is fully extended (e.g., four feet of extension). The left arm lead spool retraction spring 134 may be disposed on and/or within the left arm lead spool 130, such that the left arm lead spool 130 may rotate in a second direction in response to the left arm lead spool lead 132 being pulled when the left arm lead spool wire 131 is already fully extended, resulting in the left arm lead spool wire 131 being retracted back into the main body 110 and wrapped around the left arm lead spool 130. Again, the left arm lead spool wire catch 133 prevents the left arm lead spool wire 131 from fully retracting into the main body 110.

The right arm lead spool 140 may include a right arm lead spool wire 141, a right arm lead spool lead 142, a right arm lead spool wire catch 143, and a right arm lead spool retraction spring 144, but is not limited thereto.

The right arm lead spool wire 141 may be wrapped around a center portion of the right arm lead spool 140, such that a portion of the right arm lead spool wire 141 is threaded through the second wire aperture 113 that is disposed at a top portion of the main body 110. The right arm lead spool lead 142 may be disposed at an end of the right arm lead spool wire 141 that is disposed outside of the main body 110, and may be held in place by the right arm lead spool wire catch 143. A user may pull on the right arm lead spool lead 142 to cause the right arm lead spool 140 to rotate in a first direction about the spool rod 120, until the right arm lead spool wire 141 is fully extended (e.g., four feet of extension). The right arm lead spool retraction spring 144 may be disposed on and/or within the right arm lead spool 140, such that the right arm lead spool 140 may rotate in a second direction in response to the right arm lead spool lead 142 being pulled when the right arm lead spool wire 141 is already fully extended, resulting in the right arm lead spool wire 141 being retracted back into the main body 110 and wrapped around the right arm lead spool 140. Again, the right arm lead spool wire catch 143 prevents the right arm lead spool wire 141 from fully retracting into the main body 110.

The left leg lead spool 150 may include a left leg lead spool wire 151, a left leg lead spool lead 152, a left leg lead spool wire catch 153, and a left leg lead spool retraction spring 154, but is not limited thereto.

The left leg lead spool wire 151 may be wrapped around a center portion of the left leg lead spool 150, such that a portion of the left leg lead spool wire 151 is threaded through the third wire aperture 114 that is disposed at a top portion of the main body 110. The left leg lead spool lead 152 may be disposed at an end of the left leg lead spool wire 151 that is disposed outside of the main body 110, and may be held in place by the left leg lead spool wire catch 153. A user may pull on the left leg lead spool lead 152 to cause the left leg lead spool 150 to rotate in a first direction about the spool rod 120, until the left leg lead spool wire 151 is fully extended (e.g., four feet of extension). The left leg lead spool retraction spring 154 may be disposed on and/or within the left leg lead spool 150, such that the left leg lead spool 150 may rotate in a second direction in response to the left leg lead spool lead 152 being pulled when the left leg lead spool wire 151 is already fully extended, resulting in the left leg lead spool wire 151 being retracted back into the main body 110 and wrapped around the left leg lead spool 150. Again, the left leg lead spool wire catch 153 prevents the left leg lead spool wire 151 from fully retracting into the main body 110.

The right leg lead spool 160 may include a right leg lead spool wire 161, a right leg lead spool lead 162, a right leg lead spool wire catch 163, a right leg lead spool retraction spring 164, but is not limited thereto.

The right leg lead spool wire 161 may be wrapped around a center portion of the right leg lead spool 160, such that a portion of the right leg lead spool wire 161 is threaded through the fourth wire aperture 116 that is disposed at a top portion of the main body 110. The right leg lead spool lead 162 may be disposed at an end of the right leg lead spool wire 161 that is disposed outside of the main body 110, and may be held in place by the right leg lead spool wire catch 163. A user may pull on the right leg lead spool lead 162 to cause the right leg lead spool 160 to rotate in a first direction about the spool rod 120, until the right leg lead spool wire 161 is fully extended (e.g., four feet of extension). The right leg lead spool retraction spring 164 may be disposed on and/or within the right leg lead spool 160, such that the right leg lead spool 160 may rotate in a second direction in response to the right leg lead spool lead 162 being pulled when the right leg lead spool wire 161 is already fully extended, resulting in the right leg lead spool wire 161 being retracted back into the main body 110 and wrapped around the right leg lead spool 160. Again, the right leg lead spool wire catch 163 prevents the right leg lead spool wire 161 from fully retracting into the main body 110.

The 12 channel port housing 170 may be disposed on the main body 110, and may include a plurality of 12 channel ports 171, a 12 channel port housing door 172, and a plurality of 12 channel port housing door springs 173, but is not limited thereto.

In order to access the plurality of 12 channel ports 171 that are disposed within the 12 channel port housing 170, a user may open the 12 channel port housing door 172, which is swingedly attached to the 12 channel port housing 170 via the plurality of 12 channel port housing door springs 173.

When the plurality of 12 channel ports 171 are exposed, the user may connect an electrocardiogram (ECG) machine 10 to the retractable ECG lead device 100 by connecting each of the electrocardiogram (ECG) 12-channel leads 11 to one of the plurality of 12 channel ports 171.

The plurality of 12 channel ports 171 may be selectively connected to the left arm lead spool wire 131, the right arm lead spool wire 141, the left leg lead spool wire 151, and the right leg lead spool wire 161, such that each of the left arm lead spool wire 131, the right arm lead spool wire 141, the left leg lead spool wire 151, and the right leg lead spool wire 161 can transmit patient information (e.g., heartbeat, heart irregularities, etc.) to the ECG machine 10, thus allowing the ECG machine 10 to provide diagnostic information regarding the patient.

The electric cable 180 may be attached to the main body 110 to provide power to the retractable ECG lead device 100, if required, to provide power to the ECG machine 10, and/or to provide further connection between the retractable ECG lead device 100 and the ECG machine 10.

The present general invention concept may include a retractable electrocardiogram (ECG) lead device 100 electrically connected to an ECG machine 10, the retractable ECG lead device 100 including a main body 110, a spool rod 120 disposed within the main body 110, a left arm lead spool 130 rotatably disposed on the spool rod 120 within the main body 110, the left arm lead spool 130 including a left arm lead spool wire 131 wrapped around a center portion of the left arm lead spool 130, and a left arm lead spool lead 132 connected to a portion of the left arm lead spool wire 131 disposed outside of the main body 110, a right arm lead spool 140 rotatably disposed on the spool rod 120 within the main body 110, the right arm lead spool 140 including a right arm lead spool wire 141 wrapped around a center portion of the right arm lead spool 140, and a right arm lead spool lead connected to a portion of the right arm lead spool wire disposed outside of the main body, a left leg lead spool rotatably disposed on the spool rod within the main body, the left leg lead spool including a left leg lead spool wire wrapped around a center portion of the left leg lead spool, and a left leg lead spool lead connected to a portion of the left leg lead spool wire disposed outside of the main body, and a right leg lead spool rotatably disposed on the spool rod within the main body, the right leg lead spool including a right leg lead spool wire wrapped around a center portion of the right leg lead spool, and a right leg lead spool lead connected to a portion of the right leg lead spool wire disposed outside of the main body 110.

The retractable ECG lead device 100 may further include a plurality of 12 channel ports 171 disposed on the main body 110 to provide the electrical connection between the retractable ECG lead device 100 and the ECG machine 10 when a plurality of 12 channel leads 11 of the ECG machine 10 are connected to the plurality of 12 channel ports 171.

The left arm lead spool wire 131 may be pulled a first predetermined distance out from a first wire aperture 113 of the main body 110, such that the left arm lead spool wire 131 remains pulled out from the main body 110 at the first predetermined distance, the right arm lead spool wire 141 may be pulled a second predetermined distance out from a second wire aperture 114 of the main body 110, such that the second arm lead spool wire 141 remains pulled out from the main body 110 at the second predetermined distance, the left leg lead spool wire 151 may be pulled a third predetermined distance out from a third wire aperture 115 of the main body 110, such that the left leg lead spool wire 151 remains pulled out from the main body 110 at the third predetermined distance, and the right leg lead spool wire 161 may be pulled a fourth predetermined distance out from a fourth wire aperture 116 of the main body 110, such that the right leg lead spool wire 161 remains pulled out from the main body 110 at the fourth predetermined distance.

The left arm lead spool wire 131 may be retracted back into the main body 110 and automatically wrapped around the center portion of the left arm lead spool 130 in response to the left arm lead spool wire 131 being pulled again when the left arm lead spool wire 131 is already extended at the first predetermined distance, the right arm lead spool wire 141 may be retracted back into the main body 110 and automatically wrapped around the center portion of the right arm lead spool 140 in response to the right arm lead spool wire 141 being pulled again when the right arm lead spool wire 141 is already extended at the second predetermined distance, the left leg lead spool wire 151 may be retracted back into the main body 110 and automatically wrapped around the center portion of the left leg lead spool 150 in response to the left leg lead spool wire 151 being pulled again when the left leg lead spool wire 151 is already extended at the third predetermined distance, and the right leg lead spool wire 161 may be retracted back into the main body 110 and automatically wrapped around the center portion of the right leg lead spool 160 in response to the right leg lead spool wire 161 being pulled again when the right leg lead spool wire 161 is already extended at the fourth predetermined distance.

A purpose of the retractable ECG lead device 100 is to provide users with an innovative retractable lead box designed specifically for LIFE PACK and ZOLL lead systems. During patient assessment and trying to obtain an ECG, the wires for the ECG never fail to get tangled which requires professionals to invest effort to remedy. Even with the attention to store said wires with care, they tend to always become matted thus exhausting time and mental focus. Ingenious and practical, the retractable ECG lead device 100 offers a modern accessory that allows users to neatly store and retrieve leads, eliminating the hassle of wire management by streamlining the process. The retractable ECG lead device 100 introduces a novel retractable spool mechanism that allows users to easily pull out the leads to the desired length and lock them in place. With a simple press of a button or a small pull on the lead, the retractable ECG lead device 100 retracts the wires back into the compact storage device, neatly housing all four limb leads. With its avant-garde design, the retractable ECG lead device 100 saves both valuable time and mental energy that would otherwise be spent on the task of wire separation often associated with traditional lead storing and organization methods. By eliminating the unnecessary frustration of tangled wires, the retractable ECG lead device 100 allows users to provide prompt and efficient care, ultimately, improving patient outcomes, and as a result, may prove to be essential in the medical/therapeutic industry.

Although a few embodiments of the present general inventive concept have been shown and described, it will be appreciated by those skilled in the art that changes may be made in these embodiments without departing from the principles and spirit of the general inventive concept, the scope of which is defined in the appended claims and their equivalents.

1. A retractable electrocardiogram (ECG) lead device electrically connected to an ECG machine, the retractable ECG lead device comprising:
a main body;
a spool rod disposed within the main body;
a left arm lead spool rotatably disposed on the spool rod within the main body, the left arm lead spool comprising:
a left arm lead spool wire wrapped around a center portion of the left arm lead spool, and
a left arm lead spool lead connected to a portion of the left arm lead spool wire disposed outside of the main body;
a right arm lead spool rotatably disposed on the spool rod within the main body the right arm lead spool comprising:
a right arm lead spool wire wrapped around a center portion of the right arm lead spool, and
a right arm lead spool lead connected to a portion of the right arm lead spool wire disposed outside of the main body;
a left leg lead spool rotatably disposed on the spool rod within the main body the left leg lead spool comprising:
a left leg lead spool wire wrapped around a center portion of the left leg lead spool, and
a left leg lead spool lead connected to a portion of the left leg lead spool wire disposed outside of the main body; and
a right leg lead spool rotatably disposed on the spool rod within the main body the right leg lead spool comprising:
a right leg lead spool wire wrapped around a center portion of the right leg lead spool, and
a right leg lead spool lead connected to a portion of the right leg lead spool wire disposed outside of the main body.

2. The retractable ECG lead device of claim 1, further comprising:
a plurality of 12 channel ports disposed on the main body to provide the electrical connection between the retractable ECG lead device and the ECG machine when a plurality of 12 channel leads of the ECG machine are connected to the plurality of 12 channel ports.

3. The retractable ECG lead device of claim 1, wherein:
the left arm lead spool wire is pulled a first predetermined distance out from a first wire aperture of the main body, such that the left arm lead spool wire remains pulled out from the main body at the first predetermined distance;
the right arm lead spool wire is pulled a second predetermined distance out from a second wire aperture of the main body, such that the second arm lead spool wire remains pulled out from the main body at the second predetermined distance;
the left leg lead spool wire is pulled a third predetermined distance out from a third wire aperture of the main body, such that the left leg lead spool wire remains pulled out from the main body at the third predetermined distance; and
the right leg lead spool wire is pulled a fourth predetermined distance out from a fourth wire aperture of the main body, such that the right leg lead spool wire remains pulled out from the main body at the fourth predetermined distance.

4. The retractable ECG lead device of claim 3, wherein:
the left arm lead spool wire is retracted back into the main body and automatically wrapped around the center portion of the left arm lead spool in response to the left arm lead spool wire being pulled again when the left arm lead spool wire is already extended at the first predetermined distance;
the right arm lead spool wire is retracted back into the main body and automatically wrapped around the center portion of the right arm lead spool in response to the right arm lead spool wire being pulled again when the right arm lead spool wire is already extended at the second predetermined distance;
the left leg lead spool wire is retracted back into the main body and automatically wrapped around the center portion of the left leg lead spool in response to the left leg lead spool wire being pulled again when the left leg lead spool wire is already extended at the third predetermined distance; and
the right leg lead spool wire is retracted back into the main body and automatically wrapped around the center portion of the right leg lead spool in response to the right leg lead spool wire being pulled again when the right leg lead spool wire is already extended at the fourth predetermined distance.
